(19) United States
(12) Patent Application Publication
(10) Pub. No.: US 2008/0183157 A1
(43) Pub. Date: Jul. 31, 2008

(54) D. A. W. (Non-Invasive) Catheters

(76) Inventor: Debra A. Walters, Copiague, NY (US)

Correspondence Address: WILSON DANIEL SWAYZE, JR., 3804 CLEARWATER CT., PLANO, TX, 75025, US

(21) Appl. No.: 12/016,173

(22) Filed: Jan. 17, 2008

Related U.S. Application Data
(60) Provisional application No. 60/881,218, filed on Jan. 20, 2007.

(51) Int. Cl. A61M 27/00

(52) U.S. Cl. 604/544

(57) ABSTRACT

A male catheter device for elimination of waste from a user may include a tip section for connection to a containment bag, a cylindrical body section for connection to the tip section and a movement stopping lip positioned on the cylindrical body section and adjacent to the tip section in order to suppress undesired movement of the tip section. A female catheter device for eliminating waste from a user may include a female collection device for collecting the waist, a first strap for connection to the female collection device adapted for a thigh of the user and a second strap for connection to the female collection device adapted for the waist of the user.

### PRIORITY

The present invention claims priority under 35 USC section 119 based upon a provisional application with the Ser. No. 60/881,218 filed on Jan. 20, 2007.

### FIELD OF THE INVENTION

The invention relates to male and female external catheters and methods for making male external catheters.

### BACKGROUND

A catheter is a small-diameter, hollow, flexible tube, inserted into a body cavity, duct, or vessel to allow the passage of fluids into or out of the body, or to distend a blocked passageway such as a coronary artery or vein. Urinary catheters are inserted into the patient's urethra and thence to the bladder, and provide for elimination of urine from patients who are incontinent (lack voluntary control over their bladder) or are unable to empty their bladder. Such patients range from pregnant women to persons undergoing epidural anesthesia; persons who are paraplegic or quadriplegic; amputees; and many who are incontinent simply as a result of advancing age. Being catheterized is not only unpleasant in itself, as one can easily imagine, but also frequently has consequences which make the situation even worse.

For patients who are unable to urinate, there is no practical alternative to draining the bladder through use of an internal catheter. Long-term use of internal urinary catheters, however, can result in urinary tract or kidney infections; blood infections (septicemia); injury to the urethra; skin breakdown; bladder stones; and blood in the urine (hematuria). After many years of catheter use, bladder cancer may also develop.

More particularly, many male patients lack the ability to control urine discharge. For example, severely injured or heavily sedated male patients can lack the muscle control needed to prevent unintended urine discharge. Men suffering from incontinence also lack the ability to control urine discharge. Thus, male patients as well as men suffering from incontinence can use male external catheters to direct discharged urine away from the body and into a collection device. Once in place, the male external catheter provides a urine-impermeable barrier that directs discharged urine into a collection device protecting the user's body from urine contact.

Hospitalized patients are often unable, and typically as a consequence of a post-operative condition, to control their urinary functions. Also, those who are able to control such functions may be physically unable to use a urinal or bedpan. Therefore, patients in either of said categories are fitted with urine collection devices known as catheters. Male patients are provided with internal or urethral catheters for as long as urine volumes need to be monitored, after which time, external catheters are used. Women, however, typically continue to keep internal catheters for as long as they remain unable to use normal facilities and, as a result thereof, frequently develop severe bladder or urinary track (UTI) infections. Such UTIs are typically nosocomial infections which, it has been determined, affect approximately five percent of all hospitalized individuals. For such patients, nosocomial infections can lead to pain, disability, a longer hospital stay, or worse. In the United States alone, the treatment of nosocomial infections add billions of dollars each year to the cost of healthcare. Further, it has been determined that UTIs account for forty percent of all nosocomial infections. Accordingly, the use of internal or indwelling urethral catheter account for a large proportion of all hospital-associated infections which occur. Since a nurse, or nursing assistant, typically does not have authority to determine whether or not a patient should use a urinary catheter, the issue then becomes one of which type of catheter can be used to minimize nosocomial and other urinary track infections. It has, thereby been determined that the instance of UTIs can be reduced where an external catheter can be used in lieu of an indwelling device. However, few such female external collection devices have appeared in the art, none of which have afforded an entirely satisfactory result.

Examples of such devices appear in U.S. Pat. No. 3,601,125 to Moss; U.S. Pat. No. 4,233,978 to Hickey; U.S. Pat. No. 4,563,183 to Barrodale; and U.S. Pat. No. 4,615,692 to Giacalone. Within this limited prior art, few patents suggest a unit having a projection or element adapted for insertion into the vagina itself, these comprising U.S. Pat. No. 3,776,235 to Ratcliffe; U.S. Pat. No. 4,194,308 to Anderson; U.S. Pat. No. 4,198,979 to Cooney; said U.S. Pat. No. 4,563,183; and said U.S. Pat. No. 4,615,692.

U.S. Pat. No. 7,044,939 to Fajnszajn discloses a male external urinary catheter including a cylindrical discharge tube connected to a funnel through a bulbous zone wherein the extreme edge of the funnel is fitted with a rolled up coverette of latex rubber, polyurethane or the like that can be unfurled to encapsulate the patient's male organ, a unitary strap is provided having an connecting end unitary attached to said coverette and formed of a like material therewith and having intermediate and free end regions adapted to be looped about the coverette back upon themselves, the unitary strap including a fastener for attaching the strap upon itself without contacting the skin of a male patient.

U.S. Pat. No. 6,805,690 to Ogden discloses male external catheters having kink-resistant drainage stems as well as methods for making male external catheters having kink-resistant drainage stems. Specifically, the invention provides male external catheters having a drainage stem designed to reduce or prevent kinking that can occur during use when a drainage hose fitting and drainage hose are attached to the catheter. The invention also provides male external catheters having skin-friendly compounds and methods for making male external catheters having skin-friendly compounds. Specifically, the invention provides male external catheters designed to have skin-friendly compounds that contact the user's skin when the catheter is being used.

U.S. Pat. No. 4,233,978 to Hickey discloses an improved female catheter that is applied and used externally. It is particularly useful for the incontinent patient. The external female catheter is an extremely thin-walled device that is of substantially flat configuration that completely covers the entire vulva area of the female. The extremely thin walls expand easily and readily for temporary surges of expelled urine that increase the volume, but the thin walls return to the substantially flat configuration as the urine is drained out of the device. The device is reinforced at the edges by a thickening of the thin walls. The device is attached to the patient with an adhesive. Once attached to the female, it can remain in place for a considerable period of time and is capable of being flushed for sanitary purposes without removing it. The device is fitted with a drainage system to carry off expelled urine to a container attached to the leg.

U.S. Pat. No. 6,428,521 to Droll discloses a female external catheter device collects and then channels urine away from the human body to a collection drain. The device minimizes urinary track infections, nosocomial infections and reduces a patient's risk of infection complications. The device also reduces usage of indwelling urinary catheters. It is characterized by a ladle-like geometry in which the spoon thereof is an intra-labial collection cup and the handle is a hollow neck and nob that is secured within the vagina. Apertures with the nob enable venting of vaginal fluids thru a fluid egress channel.

### SUMMARY

A male catheter device for elimination of waste from a user may include a tip section for connection to a containment bag, a cylindrical body section for connection to the tip section and a movement stopping lip positioned on the cylindrical body section and adjacent to the tip section in order to suppress undesired movement of the tip section.

The tip section may increase in diameter, and the tip section may include a depression area.

The male catheter device my be flexible, or the male catheter device may be flexibly rigid.

The cylindrical body section may include a slit in, and the cylindrical body section may include a fastening device to fasten a first edge and a second edge formed by the slit.

The male catheter device may include an aperture, and the aperture may extend through the male catheter device.

The aperture may not extend through the male catheter device, and the aperture may be formed on the cylindrical body section.

The aperture may be formed on the tip section.

A female catheter device for eliminating waste from a user may include a female collection device for collecting the waist, a first strap for connection to the female collection device adapted for a thigh of the user and a second strap for connection to the female collection device adapted for the waist of the user.

The female catheter device may include a third strap adapted for the thigh of the user.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a perspective view of the male catheter device of the present invention;

FIG. 2 illustrates a perspective view of the male catheter device of the present invention connected to a collection bag through a collection tube;

FIG. 3 illustrates a perspective view of the male catheter device of the present invention;

FIG. 4 illustrates a cross-sectional view of the male catheter device of the present invention;

FIG. 5 illustrates a front view of the female catheter device of the present invention;

FIG. 6 illustrates a side view of the female catheter device of the present invention.

### DETAILED DESCRIPTION

The present invention includes a male catheter device and a female catheter device which may provide individuals requiring urinary catheterization with a more comfortable, reliable, external catheter, thus avoiding the problems of discomfort and infection so prevalent with internal catheterization.

Given the fact that some patients may require an internal urinary catheter despite all these risks, it is also true that patients who are simply incontinent being unable to prevent the release of their bladder would not require an internal catheter, and would surely prefer an external catheter. The present invention provides a external catheter fitted to and worn on the outside of the body, and provide watertight conduction of the urine through a collection tube to a standard medical fluids bag, which could then be emptied at appropriate intervals and reused. The present invention provides an external catheter that is comfortable and easy to wear, drains efficiently to prevent any urine backflow onto the patient, and permits smooth, watertight, and easy connection to the conducting tube and drainage bag.

Recognizing the need of incontinent persons for a more comfortable, hygienic mode of external catheterization, the present invention offers external catheterization systems for both men and women, these systems including a formfitting collection device; the conduction tubing; and a drainage bag or reservoir designed for repeated use. All the components could be manufactured of latex, plastic, a clear synthetic polymer, or surgical rubber or other suitable materials. Both the male and female catheters should fit snugly and securely, but comfortably, and that the flow of urine should be conducted efficiently into the collection tube. The male external catheter includes a formfitting sheath which may include a plastic mesh, latex, silicone or rubber, which may be fitted over the patient's penis. A suitable lubricant could be applied preliminary to the fitting of this sheath, thus allowing the sheath to rotate without damaging the patient's skin. The sheath may be perforated throughout its surface, thus allowing the patient's skin to breathe. At the distal end of the sheath, the male catheter device includes a tapered, funnel-like opening for the passage of the patient's urine, and the male catheter device may attach in watertight fashion to the collection tube. The male catheter device may include a one-way, non-kinking flow valve in the end so that no backflow of urine could occur. The male catheter device may include a strap clamp or elastic band in order to keep the male catheter device and tubing in place.

The collection tube may include a section of standard medical/surgical tubing, the length varying according to the patient's circumstances and mobility.

(Ambulatory patients may opt to wear the drainage bag affixed to one leg, and require a relatively short length of collection tubing; bedridden or wheelchair-restricted patients may prefer other arrangements and lengths of tubing.) In any event, the collection tube conducts the patient's urine to the collection or drainage bag, where it collects. The tube would connect in a watertight fitting at the neck of the drainage bag, probably fixed there by a standard tube connection at the bottom of the drainage bag would feature a clamped spout or tube that could be opened to empty the bag, then closed for reuse. All of these components could be cleaned or could be disposable with standard medical disinfecting agents and reused. The male catheter device may be changed regularly, and could be sold in inexpensive, multi-product packages or singularly.

The female catheter device may include lower two components the collection tube and the reservoir or drainage bag may be similar to those for the male catheter device.

The female catheter device would include a female collection device formed of silicone, rubber, latex, a soft and pliable plastic or other suitable material. The device would cooperate with the contours of the female anatomy, and fit snugly but comfortably in the genital region, and the female catheter device may include for attachment to the to the wearer's body with three straps, one for each thigh and a third for the waist. Again, the collecting device would conduct the urine into the collecting tube, and a one-way flow valve may be a desirable feature to prevent potential backflow. From there, the system proceeds as previously described. The female collecting device might be reusable after cleaning and disinfection or may be disposable or might be sold separately, as noted with regard to the male sheath.

The male catheter device and the female catheter device provide incontinent persons with a number of distinct and significant benefits and advantages. Foremost, by avoiding the insertion of a catheter into the patient's body, the patient would also avoid the unpleasant potential consequences of prolonged internal catheterization, urinary tract or kidney infections, blood infections, injury to the urethra, skin breakdown, bladder stones; and blood in the urine. In addition the male and female catheters devices are relatively comfortable by comparison, easy to apply or remove, and effective in providing clean and dependable service during periods of urinary incontinence, whether extended or brief. The male and female catheter devices can equally accommodate ambulatory patients, wheelchair-restricted patients, and patients confined to bed. Although internal catheters will remain a necessity in many cases, the male and female catheters of the present invention, for many patients who experience incontinence regarding their bladder function, would offer a painless alternative, and a better solution.

External urinary catheters designed for men and women who are experiencing lack of voluntary bladder control, the male and female catheter devices may solve the problem of incontinence without increasing the risk of urinary-tract infection. The catheter devices of the present invention are easily applied by hospital staff or by the patient at home and may enhance the comfort and convenience of users. The catheter devices of the present invention provide an alternative to internal catheters at an easily affordable price. For those men and women who require a catheter for incontinence but would prefer to avoid the discomfort and potential side-effects of internal catheterization, the catheter devices of the present invention provide the service, but not the side-effects.

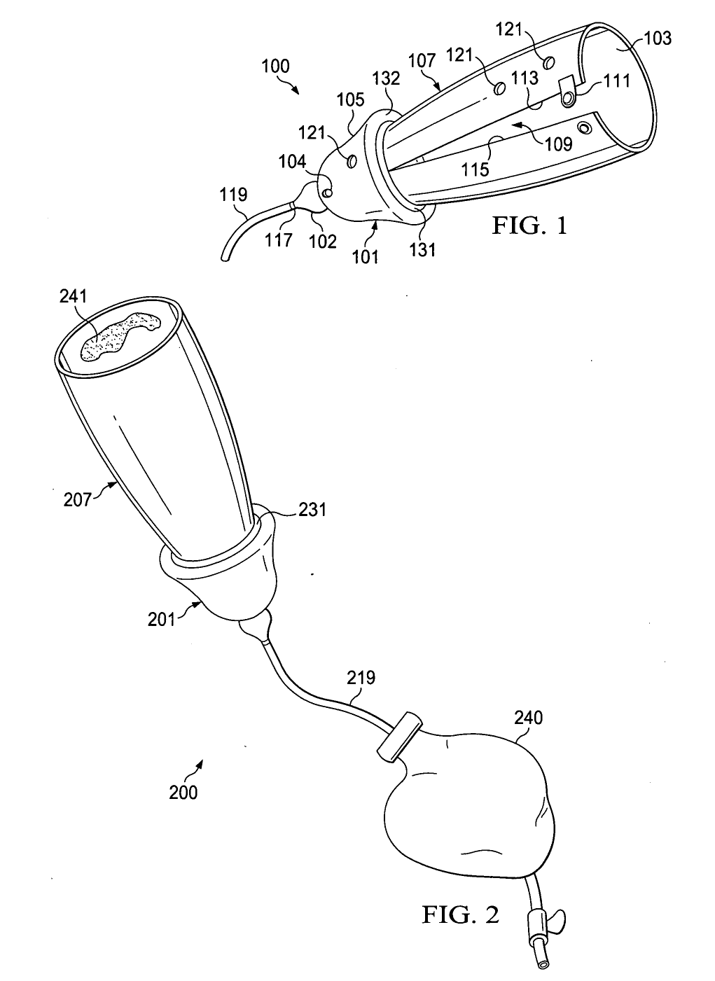

FIG. 1 illustrates the external male catheter 100 of the present invention which is flexibly rigid or flexible. The external male catheter 100 includes an increasing diameter tip section 101 and a cylindrical body section 107. The increasing diameter tip section 101 is formed at a distal end of the external male catheter 100 and is form fitted to the head of a penis of a male user of the present invention. The increasing diameter tip section 101 approximates a tapered funnel shape device and increases in diameter from the distal end to the proximate end of the increasing diameter tip section 104 and may include a depression area 105 to more accurately form fit the head of the penis. The increasing diameter tip section 101 includes a second end aperture 104 formed approximately in the center of the distal end of the increasing diameter tip section 101 to allow urine to escape from the user and the male catheter device 100. The second end aperture 104 is covered by a containment tube 119 which may include a one-way valve 117 to prevent the urine from backing up and reentering the male catheter device 100. The proximate end of the increasing diameter tip section 101 includes a movement stopping lip 131 to cooperate with the penis lip of the male penis and prevents the male catheter device 100 from prematurely being removed from the penis and allows the increasing diameter tip section 102 more closely form fit the head of the penis in order to substantially provide a seal which prevents the urine from escaping and allows substantially complete drainage of the urine through the second end aperture 104. The movement stopping lip 131 is formed on the cylindrical body section 107 which is of a lesser diameter than the movement stopping lip 131 and adjacent to the increasing diameter tip section 101 and may be separated from the increasing diameter tip section 101 by a substantially perpendicular wall 132. The cylindrical body section 107 may include a slit 109 which extends in the longitudinal direction of the cylindrical body section 107 and may extend the entire length of the cylindrical body section 107 or only a partial length of the cylindrical body section 107. The slit 109 is defined by a first edge 113 and an opposing second edge 115 and allows the easy insertion and withdrawal of the penis from the male catheter device 100. The slit 109 may be closed and the first edge 113 and the second edge 115 are brought together and held by a fastening device 111 which may be a hook and loop, snap device, button or other appropriate fastening device. The cylindrical body section 107 may include a first end aperture 103 to allow the penis to enter the male catheter device 100 and exit the male catheter device 100. The first end aperture 103 may be located at the proximate end of the male catheter device 100. Furthermore, the male catheter device may include breathing apertures 121 which may be located on the cylindrical body section 107 and/or the increasing diameter tip section 101. The breathing apertures 121 allows the skin of the penis to breathe, prevents infection and sores.

FIG. 2 illustrates another embodiment of the present invention. FIG. 2 shows the increasing diameter tip section 201, the cylindrical body section 207, the containment tube 219 to connect the increasing diameter tip section 201 to the containment bag 240 and the containment bag 240 where the urine is collected to be drained at a later time. The embodiment shown in FIG. 2 does not include a slit. To aid in insertion of the penis, lubricant 241 is positioned on the interior surface of the cylindrical body section 207 to prevent skin from being torn. The lubricant 241 may be placed on the interior surface of the cylindrical body section at manufacture or before insertion.

FIG. 2 illustrates the movement stopping lip 231 formed on the surface of the cylinder body section 207 adjacent to the increasing diameter tip section 201. The increased resistance from the movement stopping lip 231 prevents the head of the penis from prematurely moving within the increasing diameter tip section 201.

FIG. 3 illustrates the movement stopping lip 331 and the fastening device 311 to fasten opposing edges 413, 415 of the cylindrical body section 307. FIG. 3 additionally illustrates that the breathing apertures 321 do extend completely through the cylindrical body section 307. However, the breathing apertures 121 may extend through the cylindrical body section 107 as shown in FIG. 1.

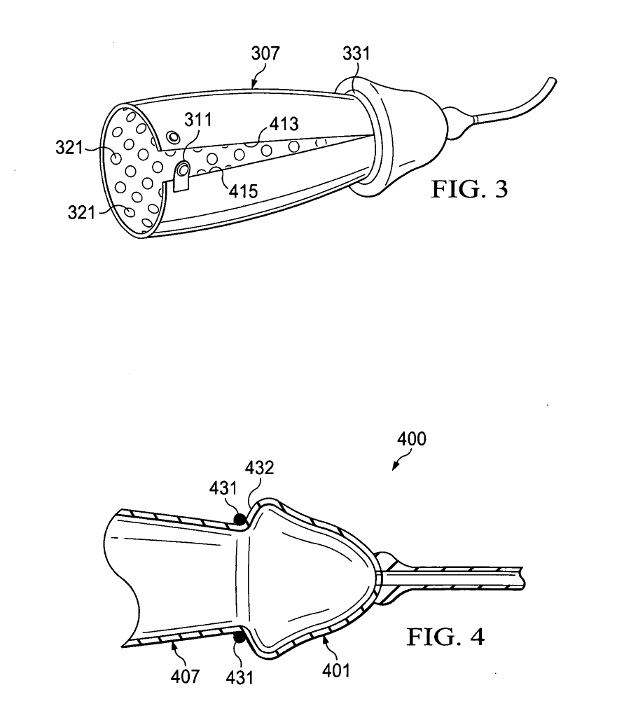

FIG. 4 illustrates a cross-sectional view of the male catheter device 400 of the present invention. FIG. 4 shows that the increasing diameter tip section 401 includes a substantially perpendicular wall 432 which is adjacent to the movement stopping lip 431 which is formed on the cylindrical body section 407.

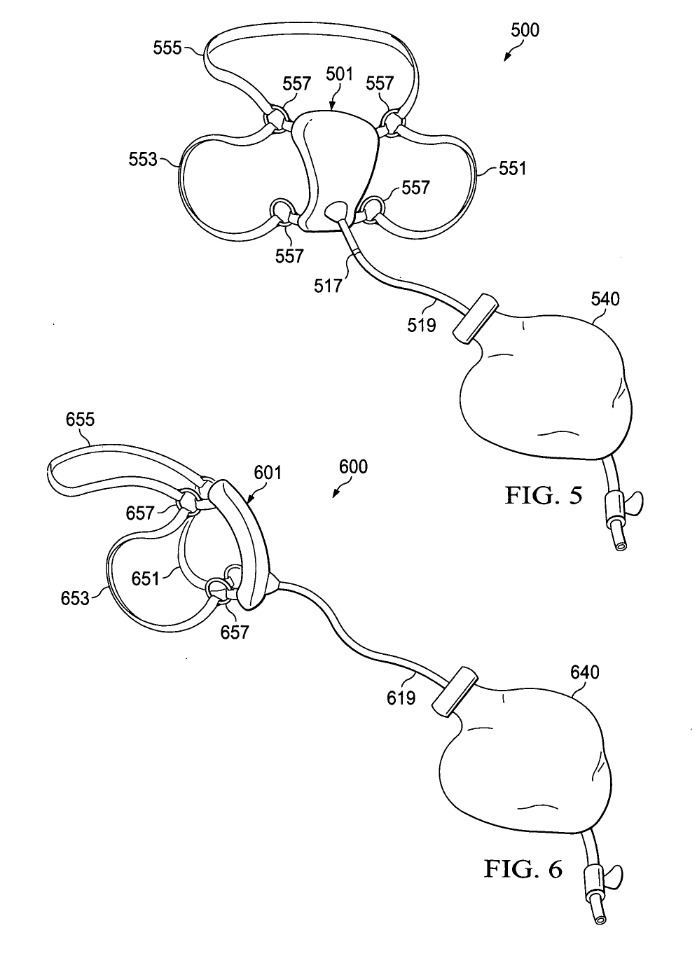

FIG. 5 illustrates a front view of the female catheter device 500 would include a female collection device 501 which may be formed of silicone, rubber, latex, a soft and pliable plastic or other suitable material. The female collection device 501 is connected to a collection tube 519 which is connected to a collection bag 540. The female collection device 501 should cooperate with the contours of the female anatomy, and fit snugly but comfortably in the genital region, and the female catheter device 501 may include for attachment to the to the wearer's body with three straps 551,553,555, a first strap 551 and a second strap 553, one going around each thigh and a third strap 555 for the waist. The straps 551, 553, 555 are connected to the female collection device 501 by fastening devices 557 which could be rings or other suitable fastening devices. The straps may be expendable by using expendable material. Again, the female collection device 501 would conduct the urine into the collecting tube 519, and a one-way flow valve 517 may be a desirable feature to prevent potential backflow. The female collecting device 501 might be reusable after cleaning and disinfection or disposable or disposable, and might be sold separately.

FIG. 6 illustrates a side view of the female catheter device 600 would include a female collection device 601 which may be formed of silicone, rubber, latex, a soft and pliable plastic or other suitable material. The female collection device 601 is connected to a collection tube 619 which is connected to a collection bag 640. The female collection device 601 should cooperate with the contours of the female anatomy, and fit snugly but comfortably in the genital region, and the female catheter device 601 may include for attachment to the to the wearer's body with three straps 651, 653, 655, a first strap 651 and a second strap 653, one going around each thigh and a third strap 555 for the waist. The straps 651, 653, 655 are connected to the female collection device 601 by fastening devices 657 which could be rings or other suitable fastening devices. The straps may be expendable by using expendable material.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A male catheter device for elimination of waste from a user, comprising:
a tip section for connection to a containment bag;
a cylindrical body section for connection to the tip section;
a movement stopping lip positioned on the cylindrical body section and adjacent to the tip section in order to suppress undesired movement of the tip section.

2. A male catheter device for elimination of waste from a user as in claim 1, wherein the tip section increases in diameter.

3. A male catheter device for elimination of waste from a user as in claim 1, wherein the tip section includes a depression area.

4. A male catheter device for elimination of waste from a user as in claim 1, wherein the male catheter device is flexible.

5. A male catheter device for elimination of waste from a user as in claim 1, wherein the male catheter device is flexibly rigid.

6. A male catheter device for elimination of waste from a user as in claim 1, wherein the cylindrical body section includes a slit.

7. A male catheter device for elimination of waste from a user as in claim 7, wherein the cylindrical body section includes a fastening device to fasten a first edge and a second edge formed by the slit.

8. A male catheter device for elimination of waste from a user as in claim 1, wherein the male catheter device includes an aperture.

9. A male catheter device for elimination of waste from a user as in claim 8, wherein the aperture extends through the male catheter device.

10. A male catheter device for elimination of waste from a user as in claim 8, wherein the aperture does not extend through the male catheter device.

11. A male catheter device for elimination of waste from a user as in claim 8, wherein the aperture is formed on the cylindrical body section.

12. A male catheter device for elimination of waste from a user as in claim 8, wherein the aperture is formed on the tip section.

13. A female catheter device for eliminating waste from a user, comprising:
a female collection device for collecting the waist;
a first strap for connection to the female collection device adapted for a thigh of the user;
a second strap for connection to the female collection device adapted for the waist of the user.

14. A female catheter device for eliminating waste from a user as in claim 13, wherein the female catheter device includes a third strap adapted for the thigh of the user.
